(19) United States
(12) Patent Application Publication
(10) Pub. No.: US 2005/0244526 A1
(43) Pub. Date: Nov. 3, 2005

(54) Use of a preparation produced from plant seedlings, enriched with electrolyte

(76) Inventors: Norbert Fuchs, Mariapfarr (AT); Peter Kossler, Mariapfarr (AT)

Correspondence Address: FULBRIGHT & JAWORSKI L.L.P., 600 CONGRESS AVE., SUITE 2400, AUSTIN, TX, 78701, US

(21) Appl. No.: 10/516,789

(22) PCT Filed: May 19, 2003
(86) PCT No.: PCT/AT03/00145

§ 371 Date: Jul. 5, 2005

(30) Foreign Application Priority Data
Jun. 7, 2002 (AT) A 872/2002

(51) Int. Cl. A61K 35/78

(52) U.S. Cl. 424/776

(57) ABSTRACT

Described is the use of an agent prepared from plant seedlings enriched with electrolytes for the production of a pharmaceutical preparation aimed at the proliferation of T-lymphocytes, the reduction of cholesterol and the reduction of LDL. In general, rejuvenation of the immune system was shown to result from the use according to the invention. In particular, an improved vigilance and increased reagibility of the immune system were noted.

The invention relates to novel uses of agents prepared from seedlings enriched with electrolytes.

While the positive influence of regularly consumed fruits, vegetables and roughage on health and the immune system is uncontested, the uptake of individual, isolated vitamins frequently yields contradictory results in clinical assays and intervention studies.

It is known that demand-adequately dosed and combined micronutrient complexes exert positive influences on the immune system of HIV-positive patients, being particularly able to raise the quotient from T helper cells and T suppressor cells (N. Fuchs et al.: WMW 1996; 145:483-493. I. Guth et al.: J. f. Ortho Med. 5; 4 (1997) 325-348).

By contrast, the isolated uptake of individual nutrients such as, e.g., antioxidative beta-carotene will result in elevated lung cancer incidences and higher mortality rates in smokers (ATBC Study: New England Journal of Medicine 1994; 15: 1029-1035). Another study revealed an increase in the risk of strokes after the ingestion of isolated synthetic vitamin E.

Laboratory vitamins, thus, apparently do not always exhibit the positive effects that are expected from vital substances derived from fruits and vegetables.

An essential reason for the different actions of “chemical” and “natural” vitamins in man may reside in the complexity of the human cell metabolism. From biochemistry, some 50,000 different metabolic reactions have been known to date. Each of these reactions will require a special vitamin, trace element or any other vital substance to proceed in the optimum manner. In order to remain healthy, the human body, thus, does not need individual vitamins and trace elements but a plurality of mutually supporting and regenerating vital substances (vitamins, trace elements, essential fatty acids, so-called secondary plant substances). As numerous dietetic studies have shown, the human metabolism, particularly that of the immune system, calls for the complex interplay of cellular enzyme activators, mutually regenerating antioxidative systems, highly unsaturated fatty acids for the regeneration of cellular biomembranes as well as interstitial connective tissue modulators.

Comparative studies of the vegetable and human cell metabolisms have shown major analogies in the metabolisms of proteins, fats and carbohydrates. Unlike human cells, plant cells are, however, able to endogenously synthesize, via photosynthetic processes, the organic reduction equivalents (e.g. vitamins) increasingly required for growth and regeneration (A. E. Harmuth-Hoene, A. E. Bognar et al.: Der Einfluss der Keimung auf den Nährwert von Weizen, Mungbohnen und Kichererbsen, Z. Lebensm. Unters. Forsch. 1987, 185; 286-393). The increased endogenous synthetic performance of germinant cereal cells for antioxidative vitamins and highly unsaturated fatty acids, thus, is supposed to be the biochemical expression of an elevated demand in the context of reductive constitutional and regeneration processes. In the context of numerous test series, it was feasible to enrich germinant cereal seeds with mineral essential trace elements (J. Lintschinger, N. Fuchs et al.: Uptake of various trace elements during germination of wheat, buckwheat and quinoa; Plant Foods for Human Nutrition 50: 223-237, 1997). The moderate supply of essential trace elements, thus, helped increase both the enzymatic synthetic performance in forming organic vitamins and the content of organically bound trace elements (J. Lintschinger, N. Fuchs et al.: Selenium Enriched Sprouts. A Raw Material for Fortified Cereal-Based Diets; J. of Agricultural and Food Chemistry, 2000: 48, 11: 5362-5368).

The germination of plant seeds in electrolytic solutions for the production of electrolyte-enriched seedlings is, for instance, described in EP 0 770 324 A and EP 0 799 578 A.

The object of the present invention consisted in providing novel applications for such agents produced from seedlings enriched with electrolytes.

The object of the present invention, therefore, resides in the use of an agent prepared from plant seedlings enriched with electrolytes for the production of a pharmaceutical preparation aimed to proliferate T-lymphocytes in non-immune-suppressed persons. It was shown in a surprising manner that said agents were able to provide positive effects not only in immune-suppressed persons having pathologic values in respect to their cellular immune systems, but also in non-immune suppressed persons. In the context of the present invention it was, however, demonstrated that—unlike with the treatment of immune-suppressed individuals, which goes hand in hand with an increase in the quotient from T helper cells and T suppressor cells (cf. EP 0 799 578 A)—that quotient in non-immune-suppressed individuals develops into the opposite direction, that is to say rather towards T suppressor cells.

In a preferred manner, the present invention, therefore, generally contemplates the proliferation of CD3+ specific immune cells and, in particular, CD3+/CD4+ specific immune cells (helper cells), CD3−/CD16,56+ specific immune cells (natural killer cells), CD4+/CD45RA+ specific immune cells (naive T cells) and CD3+/CD8+specific immune cells (suppressor cells).

In general, rejuvenation of the immune system was shown to result from the use according to the invention. In particular, an improved vigilance and increased reagibility of the immune system were noted.

It was, furthermore, shown that the agents prepared from plant seedlings enriched with electrolytes according to the invention could also be used for the production of a pharmaceutical preparation aimed to lower cholesterol concentration in blood (i.e., induce reduced cholesterol concentration in blood).

Furthermore, agents of this type are suitable for the production of a pharmaceutical preparation aimed to reduce low-density lipoprotein (LDL) concentration in blood (i.e., to induce reduced LDL concentration in blood).

The uses according to the invention have turned out to be particularly successful in the geriatric field, as will be impressively proved by the results from a medical study, which are indicated in the Examples.

In accordance with the invention, the pharmaceutical preparations are preferably provided in food form in order to render the prophylactic application as easy as possible for the target individuals.

On account of the findings according to the invention, the present invention also relates to the use of the electrolyte-enriched seedling agents for the production of a pharmaceutical preparation aimed to prevent atheroscleroses, myocardial infarcts and/or apoplexies.

In doing so, it was all the more surprising that it was the cellular immune response that could be specifically influenced by the uses according to the invention, and not the humoral immune response. Furthermore, no significant effects on the red blood count (erythrocytes, hemoglobin, etc.), on the blood chemistry (except for cholesterol and LDL) or the thrombocyte count by the administration according to the invention of the electrolyte-enriched seedling preparation could be detected. Even clinical parameters like weight, temperature, heart rate, blood pressure and ECG remained unaffected.

Preferred electrolyte-enriched seedlings that may be used according to the present invention are the seedlings disclosed in EP 0 770 324 A as well as the combined preparations described in EP 0 799 578 A.

In a preferred manner, the seedlings according to the invention should have a content of one or several electrolytes, preferably zinc, iron, potassium, magnesium, copper, manganese, strontium, selenium, molybdenum, chromium, arsenic, vanadium and/or cobalt ions, elevated by at least 10 to 20%, preferably at least 1.5 to 3 times and, in particular, at least 5 to 10 times as compared to that of conventionally germinated seeds (i.e., in conventional tap water).

Conventional seed germination, in which the seeds are placed in distilled water or tap water for germination, always involves partially considerable losses of such nutritionally relevant components. Those losses were due both to the beginning metabolic process of the plant seedling itself and to the nature of the swelling agent water, which caused additional electrolyte leaching of the seedling, since, unlike in the resting state (seed), the husk of the seedling is, in fact, susceptible to electrolyte leaching.

It has, furthermore, been shown that the electrolyte-enriched seedlings used according to the invention not only have higher concentrations of mineral substances, but on account of their elevated content of mineral substances also are generally improved in terms of ingredients, for instance exhibiting elevated vitamin contents.

A preferred mode of production of the seedlings according to the invention consists in that the germinative seeds are introduced into an electrolyte solution and the seedlings are incubated in the electrolyte solution at a suitable temperature for a period of time sufficient to obtain an enrichment of electrolytes in the seedlings.

When using an electrolyte solution, that is a solution containing an increased ion concentration as opposed to conventional germination solutions (tap water or distilled water or sterilized water), the electrolyte losses occurring during germination can be compensated for or even turned into the opposite by an electrolyte flow from the germination solution into the seedlings. Thus, seedlings can form which partially even have electrolyte contents increased as against those of the seeds.

By electrolyte solution, an aqueous solution supplemented or enriched with one or several electrolytes as defined below is preferably understood.

The ion concentration of the electrolyte solution may be at least by 10 to 20% higher than that of conventional tap water, the ion concentration of the electrolyte solution at least in terms of iron and/or copper and/or manganese and/or strontium and/or lithium and/or molybdenum ions preferably being two times, in a particularly preferred manner at least five times and, in particular, at least ten times higher than that of conventional tap water.

It goes without saying that the suitable temperature for carrying out germination varies as a function of the type of seed. In principle, the germination temperature described in the prior art for the respective type of seed is to be applied also with the method according to the invention. Preferably, this temperature ranges between 10 and 50° C., particularly between 20 and 30° C.

The period of time necessary to obtain sufficient enrichment of electrolytes in the seedlings likewise differs with the type of seedling, and it is also a function of what electrolytic values are sought in the seedling. In this case too, germination periods described in the prior art serve as guide values for certain types, germination therefore being preferably carried out for a period of approximately 12 to 120 hours and, in particular, approximately 60 to 100 hours.

It goes without saying that both the germination temperature and the germination time are readily optimized by the skilled artisan by simple experiments for every system and for certain types may definitely also be below or above the guide values referred to above.

Preferred seedlings according to the invention comprise seedlings of current vegetable foods and, in particular, seedlings of leguminous plants and cereal seeds. Particularly preferred seedlings are, therefore, wheat, buckwheat, quinoa, mung bean, fenugreek, radish, alfalfa, corn, pumpkin, walnut, rye, barley, rice, adzuki bean, pea, millet, palm, oats, chick-pea, cress, linseed, lentil, mustard, sesame, soybean, sunflower, amaranth seedlings and mixtures of these seedlings.

The electrolyte solution may contain 1 mg/l or more, preferably 10 mg/l or more, particularly 50 mg/l or more, of zinc and/or iron and/or potassium and/or magnesium ions, 0.5 mg/l or more, preferably 5 mg/l or more, particularly 25 mg/l or more, of copper and/or manganese and/or strontium and/or lithium ions, 0.1 mg/l or more, preferably 1 mg/l or more, particularly 5 mg/l or more, of selenium and/or molybdenum and/or chromium and/or arsenic and/or vanadium and/or cobalt ions, with the proviso that the ion concentration of the electrolyte solution differs from the ion concentration of tap water by at last 10 to 20% in at least one ion species.

A particularly preferred electrolyte solution contains at least 0.5 mg/l copper and/or 1 mg/l zinc and/or 0.1 mg/l cobalt and, preferably, at least 0.1 mg/l molybdenum and/or 0.5 mg/l lithium and/or 1 mg/l selenium and/or 1 mg/l vanadium ions.

After their production, the electrolyte-enriched seedlings can as a function of their purpose of use be washed, dried and optionally further processed to be suitable for selling. Particularly preferred is the processing of the seedlings according to the invention to fresh food, bread spreadings, bakery products, soups or snack-like foods or food supplements in the form of mueslis, chewing tablets, capsules or liquids.

For the uses according to the invention, the seedlings are preferably employed in combination with micronutrients. Preferred micronutrients are polyunsaturated fatty acids, natural carotenoid mixtures, germ extracts, natural anthocyan mixtures, natural tocopherol and tocotrienol mixtures, vitamins and coenzymes, essential and nonessential amino acids, mineral substances and mixtures thereof.

Another object of the present invention in the sense of the claims is, of course, the non-therapeutic field.

In the following, the invention will be explained in more detail by way of the following medical study, to which it is, of course, not limited.

### EXAMPLE

Medical report on the use of a micronutrient concentrate containing preparations of electrolyte-enriched seedlings for immunomodulation in geriatrics.

Indication:

Nutritional supplementation of residents of a geriatric institution including a survey of the effects on the immune system.

Test Medication:

Based on dried electrolyte-enriched seedlings, particularly wheat seedlings, having high contents of endogenous vitamin, essential fatty acid and trace element complexes, a dietary food PMN® micronutrient concentrate was used in the context of the present trial (“immunostabilizing factor” ISF® (integrated micronutrient concentrate PMN® vis vitalis AG/Austria) versus placebo: suspendable powders in four flavors (tomato, garlic-asparagus, vegetable, mushroom). Administered daily over three months, dissolved in hot water, in soup form.).

PMN® stands for Pan Molecular Nutrients, expressing the completeness of the nutrient complexes as against synthetic combinations of vitamins and trace elements.

Trial Sequence:

Screening phase
blood taking of all parameters
2 months supplementation
influenza vaccination in week 8
1 month supplementation
blood taking of all parameters
2 months follow-up
   Target Quantities:
I) Immunology
1) phenotyping of lymphocytes by FACS
2) testing of lymphocyte function (IL-2, IL-2R) lymphocyte activatability in vitro
3) antibody titer after influenza vaccination
   Safety Parameters:
I) laboratory
II) clinical parameters
III) registration of undesired effects

The primary objectives consisted above all in the collection of parameters accepted and relevant in classical medicine, by means of which the effects of the test substance on the immune system can be evaluated. Since it was a micronutrient preparation in the form of a supplement to daily nutrition which was to be assessed, a patient population with as homogenous a distribution of the factor nutrition as possible was looked for. It was found in the form of residents of a geriatric institution, since all persons participating in the trial were looked after by the same canteen, thus entailing no falsifications of the results.

The effects of the test substance on the immune system were evaluated in terms of cellular and humoral immunity of man by way of parameters taking into account both. They included: leucocyte phenotying by FACS, in vitro activation of T lymphocytes including the determination of IL-2 and IL-2r as well as antibody titer determination after influenza vaccination in the course of the trial.

Description of Patient Collective:

Patients of both sexes, who had been residing at the geriatric center for at least 3 months were recruted. A written declaration of consent was procured from the patient or his/her trustee.

106 persons were randomized. 54 in the verum group and 52 in the placebo group.
Verum: 45 females, 9 males.
Placebo: 44 females, 8 males.

The average patient age was 85 years (62-98) in the verum group and 85.5 years (65-98) in the placebo group. The age and sex distribution corresponds to demographic studies of this age population. These distribution patterns do not significantly change in respect to the patients defined for the “per protocol analysis” as far as age and sex are concerned. Patients having consumed at least 50% of the test agent for at least 80% of the days of therapy were defined as such. The verum group, thus, included 40 (32 female and 8 male) and the placebo group 42 (35 female and 7 male) persons.

I) Target Parameters

1) Leucocyte Phenotyping:

This was carried out by means of a standardized. FACS method in agreement with good laboratory practice.

The surface markers CD2+/CD3−, CD2+/CD3+, CD3+, CD3+/CD4+, CD3+CD8+, CD3−/CD16,56+, CD3+/CD16,56+, CD19, CD3+/HLA-DR+, CD4/CD45RA+, CD4+/CD45RO+, CD8+/CD38+ were determined.

The individual sub-populations can be described and quantified by the aid of surface markers on lymphocytes. Lymphocytes are a fraction of leucocytes. They were taken and evaluated during blood taking at the beginning and at the end of the trial.

Leucocytes:

No significant difference (p=0.468) is found when comparing the initial values of the two groups.

The statistic evaluation of the changes within the groups relative to each other shows a significance in favor of the verum group of p=0.03.

From a medical point of view, a proliferation of leucocytes may be indicative of an infect. A comparison of this single parameter with the results from blood sedimentation, CRP and the recording of antipyretic drugs/antibiotics and fever days in combination of all these parameters allows for the conclusion that leucocyte proliferation in the verum group has not been caused by an elevated incidence of infects.

Lymphocytes:

A comparison of the initial values is insignificant with p=0.989.

A significance of p=0.034 at a comparison of the group changes beginning-end shows lymphocyte proliferation in the verum group. Viewed in combination with the other collected parameters, an influence by the test substance is to be assumed from a medical-hematologic point of view. This is confirmed in that no other causes (infects etc.) can be verified in the clinical sense from the combination of all available data (see above).

The lymphocytes represent the immune system. Distinction is made between T and B lymphocytes. T-Ly represent cellular immunity, while B-Ly represent humoral immunity (production of immunoglobulins). In earlier studies (see introduction), effects of the test substance on the cellular immune system were observed. Lymphocytes were sub-typified by FACS investigation.

B-Lymphocytes:

The surface marker CD-19 is representative of the population of B-Ly.

Changes occur neither in the verum group (p=0.216) nor in the placebo group (p=0.509) in the trial process.

An evaluation of the processes compared to each other does not show any significance either (p=0.856).

Since B-Ly are immunoglobulin-producing cells, their evaluation was discussed in combination with laboratory data from electrophoresis:

No medically interpretable changes were observed both in the total protein amount and in the individually evaluated subgroups (Alb, α1, α2, β and γ globulins). Considering all of the parameters representing the humoral immune system in combination, no change in the sense of a hematologic-immunologic relevance occurs in either of the two groups.

Cellular Immunity:

The population of T-Ly was determined by the aid of the surface marker profiles CD2+/CD3+, CD2+/CD3− and CD3+. Additional fractionations of the “helper cells” (CD3+/CD4+), “suppressor cells, (CD3+/CD8+), “natural killer cells” (CD3−/CD16,56+), “activated T-Ly” (CD3+/HLA-DR+) and “cytotoxic T-Ly” (CD8+/CD38+) were used to exactly describe cellular immunity.

T-Lymphocytes:

CD3+ defines T-Ly in general. Since in immunology a cell type is not localized by a single marker but by a combination of the same, also CD2+/CD3+ was determined in order to represent the population of T-Ly in as precise a manner as possible.

No significant differences in the initial values were observed in the evaluation of the two lymphocyte profiles.

CD3+

A significant increase is observed in the verum group during the process (p=0.004). A comparison of the groups likewise is significantly in favor of verum, with p=0.023.

CD2+/CD3+

Again, a significant increase in the verum group (p=0.011) and a significance of p=0.037 at a comparison of verum and placebo are observed.

From this exact definition of T-Ly and a statistic significance in both populations, it may be concluded that the test substance has caused changes in these cells.

Helper Cells CD3+/CD4+

With p=0.014 within the process, and p=0.093 at a group comparison, an effect in favor of verum is likewise demonstrated in this sub-population.

Suppressor Cells CD3+/CD8+

With p=0.003, a significant increase is observed within the verum group. With p=0.061, no significance is noted at a group comparison.

Ratio CD4+/CD8+

With p=0.019, this value has significantly dropped in the verum group during the process. At a group comparison, no significant difference is observed (p=0.979).

Natural Killer Cells CD3−/CD16,56+

With p=0.026, a significant increase in these cells is observed in the verum group. A group comparison results in a p-value of 0.509, thus being insignificant.

T-lymphocytes in Activated form CD3+/HLA-DR+

These cells represent that portion which has been converted into an activated form by an internal or external stimulus. Significances appear neither within the group processes nor at a direct group comparison.

Cytotoxic T-Lymphocytes CD8+/CD38+

Significant increases are to be noted both in the verum group (p<0.001) and in the placebo group (p=0.002). With p=0.089, the group comparison is in the non-significant range.

Representative T-Ly markers of the “aged” immune system The influence of the test substance on the immune system of the aged was described by the two parameters evaluated separately.

“Naive Cells” CD4+CD45RA+

When comparing the two groups, a significant rise of p=0.032 is observed in favor of verum. The increase within the verum process shows a p-value of 0.026.

Cells with “Memory Function” CD45RO+

The verum group notes an increase of p=0.037, no significant difference occurred between the groups.

2) Lymphocyte Activity:

To test the lymphocyte activity, sufficient quantities of cell material from each patient were deepfrozen at −70° C. for the times beginning and end. All samples were subjected in common according to good laboratory practice guidelines to processing for the following parameters:
number of activatable lymphocytes
number of interleukin-2 receptor (IL-2r)
interleukin-2 (IL-2)
   Number of Activatable Lymphocytes

Exactly 3000 lymphocytes were examined in each case. The data relate to the percentages of cells that are activatable in vivo. A significant rise of p<0.001 was observed in both groups. The p-value at a group comparison is 0.095.

Interleukin-2 Receptor

The average number of interleukin-2 receptors per 1000 cells was determined. A p<0.001 was found in the process of both groups. A comparison does not yield any significance, with p=0.488.

Interleukin-2

The values are represented as a factor. It is determined by what multiple the IL-2 production rises upon stimulation as compared to the “beginning”. A value of p=0.054 is found in the verum group. In the placebo group p=0.045. A comparison between the two groups reveals p=0.102.

3) Antibody Titer After Influenza Vaccination

One month following influenza vaccination, the sera were examined for antibodies against the strains Caledonia, Panama and Yamanashi.

Caledonia

With p<0.001, increases were significant in both groups. With p=0.745, the group difference was in the nonsignificant range.

Yamanashi

Besides significant group rises (p<0.001), a significance of p=0.042 is observed with placebo.

Panama

The assessment of this parameter can be judged only to a limited degree because of a significance of p=0.007 in favor of placebo with the initial values.

Concise Assessment of All Collected Immune Parameters

In the following section, the scientifically verified facts underlying the trial are compared with the results.

From investigations carried out during the past years it is known that the immune response decreases with increasing age. This applies, in particular, to the fraction of T lymphocytes.

The result of an analysis of the cellular immunity carried out by way of T lymphocytes in the present trial is that the group treated with verum exhibits a significant rise in these cells, with p=0.032 (CD3+) and p=0.037 (CD2+/CD3+). The total lymphocyte count likewise rises significantly with p=0.034.

Both the clonal proliferation and the maturation process of the lymphocytes decrease with increasing age. The ratio of immature cells to mature cells shifts towards immature forms. The equilibrium of suppressor to helper lymphocytes changes towards helper cells.

Results in the verum group have demonstrated a significant proliferation of CD3+/CD4+ (helper) with p=0.014, and CD3+/CD8+ with p=0.003. The changes of the T4/T8 ratio with 0.019 in the sense of a value decrease show a relative proliferation of suppressor cells. By contrast, no significance is observed in the placebo group.

It is, furthermore, known that besides a reduction of suppressor cells at an advanced age, these will also exhibit a reduced cytotoxic capacity.

When assessing the CD8+/CD38+ cytotoxic T cells in the presently available data material, an increase in both groups (verum: p=<0.001, placebo: p=0.002) is found. The difference between the groups is not significant with p=0.089, yet shows a more distinct growth in the verum group. The “natural killer cells” (CD3−/CD16,56+) have significantly increased in the verum group (p=0.026).

Naive (CD45RA+) and memory T cells (CD45RO+) during progressing life are subject to an increasing weighting of the memory function. This is related to a decrease of the “vigilance” of the aging immune system. With a p-value of 0.032, the naive T cells of the verum group are significantly higher than placebo at the end of the trial.

The phenotypical changes mentioned and referred to in the literature result in a reduced lymphocyte function with age. A reduced tendency to proliferation after antibody stimulation is equally detectable as a lower cytokin production.

In the present trial, the expression of IL-2r and the production increase in IL-2 were measured in addition to in vitro stimulation. Since the immune system is subject to very complex influences and reacts accordingly, very large standard deviations are found in the collected parameters.

The median of the percentage of activatable cells at the end of the study is 32% (2-61) in the verum group and 26% (10-52) in the placebo group. The evaluation of IL-2r per cell shows a median of 7378 (2622-28659) in the verum group and 5884 (2927-30732) in the placebo group. The increase in the IL-2 production, based on 1000 cells, exhibits a very high standard deviation and, with 0.102, is insignificant at a group comparison.

When summarizing all of the parameters listed, a positive influence of verum on phenotypical leucocyte changes in the sense of a proliferation of the cells of the cellular immune response is revealed. If the activity parameters (IL-2, IL-2r) are based on the number of activatable lymphocytes, a change in favor of verum is revealed also in this case.

By contrast, no changes are to be recognized in the field of humoral immunity. The population of B-lymphocytes does not show clear tendencies in any of the groups. This also holds for the related globulin analysis.

In the analysis of the influenza antibody titer investigated, a significant increase in the specificity “Yamanashi” is observed in the placebo group. “Panama” cannot be evaluated because of its significant initial differences. “Caledonia” is insignificant at a group comparison.

From a hematological point of view it can, thus, be said that the test substance has a positive influence on the cellular immune system in aged persons.

Overall Assessment:

The assessment of the red blood count is likewise applied as a parameter for the evaluation of the quality of life. It is based on the fact that an increased oxygen enrichment of the blood will result in enhanced cognitive performances. A correlation of the changes in the red blood count with the present test results is feasible, yet not proving in the present magnitudes.

Safety Parameters:

I) Laboratory Parameters

Blood Count:

The recovery of sufficient leucocytes was essential to the immunologic assays. The description of the individual parameters was treated in the chapter “immunology”.

Thrombocytes:

No changes at all are perceptible both in the verum and in the placebo groups. Medians and mean values are in the normal range at the beginning and at the end.

Red Blood Count:

Erythroctes

No significances are found. All ascertained values are within the standardized normal range.

Hemoglobin:

There are no significant differences between the groups and the trial times. Median and mean value remain stable in the verum group. The median in the placebo group changed from 13.1 to 12.8. Likewise, a drop of the mean value from 12.94 to 12.73 is to be noted.

Hematocrit

A significant drop in value is found in the placebo group during the trial process (p=0.002).

MCV

A significant drop in value occurs in both groups (p<0.001 verum and placebo). This drop is more pronounced in the placebo group.

MCH

No significances are displayed. The median is very stable in the verum group with 28.9 (before) and 28.85 (after). In the placebo group it changes from 29.4 to 28.5.

MCHC

A significant rise during the process is to be noted in both groups (verum p=0.025, placebo p=0.003).

Iron:

This parameter was added to the blood count because of its context to hemoglobin synthesis. It was the free iron level which was measured. In the verum group, a marked drop is observed during the trial with a p-value of 0.075.

Bilirubin

This value was also recorded because of its proximity to the red blood count. In the verum group a significant drop is observed with p=0.017.

Overall Assessment of the Present Parameters of the Red Blood Count:

Hemoglobin, hematocrit and MCV partially drop significantly in the placebo group. The iron level in the verum group suffers a near-significant drop. From a summarization of all ascertained findings, a positive effect of the test substance on the red blood count is apparent.

Blood Chemistry:

Iron and bilirubin have already been listed with the red blood cout. Therefore, only those parameters where significant changes have occured are indicated. The remaining parameters can be taken from the biometric report in a descriptive manner.

Creatinine

With p=0.006, a significant reduction of this parameter is shown in the placebo group.

GGT

A significant increase of p=0.049 is found in the verum group.

LDH

A significant rise of p=0.01 is shown in the placebo group.

Cholesterol

The verum group experiences a significant drop of p=0.002.

LDL

A reduction of this parameter with p<0.001 was evaluated in the verum group.

Albumin

p=0.005 at a drop in the verum group.

α2-Globulin

Significant drops occur both in the verum and in the placebo groups. p=0.018 with verum and p=0.026 with placebo.

Blood Sedimentation

After 1 hour:

A significant drop is to be recorded in both groups.
Verum: p=0.044; placebo: p=0.012
   After 2 hours:

Again, a significant drop of p=0.027 is found for placebo. The verum group is not significant, with p=0.09.

Interpretation of Safety Data from a Medical Point of View:

What is striking is the drop of cholesterol and low-density lipoproteins (LDL) in the verum group. An increase in LDL is considered as an internal risk factor for atherosclerotic diseases. An improvement in this parameter may be interpreted as a benefit to the patients. An assertion as to the other significantly changed parameters (creatinine, GGT, albumin, α2-globulin and LDH) is not possible due to a lack of conforming tendencies with clinically related parameters.

II) Clinical Parameters

Weight:

Comparison of median screening to follow-up after 5 months: The median in the verum group rises from 67 to 68.5 kg. In the placebo group, it drops from 59.5 to 57.5 kg.

Body Mass Index:

The median in the verum group was 25.95 at the beginning and 26.3 at the end of the trial. That of the placebo group was 24.4 before and 24.1 after completion of the observation period. There is, thus, no significant change within or between the two groups.

Temperature:

All medians ascertained in both groups are at all times at 36.2-36.3 degree C and clinically correspond to normal body temperature.

Heart Rate:

The medical evaluation of all mean values and medians at all times shows normofrequent and nonrelevant data.

Blood Pressure:

Diastolic:

At all times and in both groups no values of clinical relevance.

Systolic:

As with the diastolic value, no changes in the processes.

ECG:

Recordings to be Designated as “Pathologic”:

At a comparison screening to end of study after 3 months, the number in the verum group changes from 19 to 15. In the placebo group it changes from 22 to 16.

III) Registration of Undesired Effects

There are no reports on any undesired effects. The “serious adverse event reports” relating to the patients deceased during the trial were passed on to the ethics commission. Nor can any undesired effects be derived from the medical interpretation of the laboratory and clinical parameters. Screening of the collected ICD-9 codes for the newly occurring diseases corresponds to the normal incidence and prevalence in the geriatric group of persons examined.

1-13. (canceled)

14. A method comprising:
obtaining an agent prepared from plant seedlings enriched with electrolytes comprised in a pharmaceutical preparation; and
administering the preparation to a non-immune-suppressed person;
wherein the administration of the preparation results in proliferation of T-lymphocytes in the person.

15. The method of claim 14, wherein administration of the preparation results in proliferation of CD3+-specific immune cells in the person.

16. The method of claim 14, wherein administration of the preparation results in proliferation of CD3+/CD4+-specific immune cells (helper cells) in the person.

17. The method of claim 14, wherein administration of the preparation results in proliferation of CD3−/CD16,56+-specific immune cells (natural killer cells) in the person.

18. The method of claim 14, wherein administration of the preparation results in proliferation of CD4+/CD45RA+-specific immune cells (naive T-cells) in the person.

19. The method of claim 14, wherein administration of the preparation results in proliferation of CD3+/CD8+-specific immune cells (suppressor cells) in the person.

20. The method of claim 14, wherein administration of the preparation results in reduction of blood cholesterol concentration in the person.

21. The method of claim 20, wherein administration of the preparation results in reduction of low-density lipoprotein (LDL) concentration in the person.

22. The method of claim 14, wherein administration of the preparation results in a reduced probability of atherosclerosis, myocardial infarct, and/or apoplexy.

23. The method of claim 14, wherein the person is geriatric.

24. The method of claim 14, wherein the pharmaceutical preparation is administered in food form.

25. A method comprising:
obtaining an agent prepared from plant seedlings enriched with electrolytes comprised in a pharmaceutical preparation; and
administering the preparation to a person;
wherein administration of the preparation results in reduction of blood cholesterol concentration in the person.

26. The method of claim 25, wherein administration of the preparation results in reduction of low-density lipoprotein (LDL) concentration in the person.

27. The method of claim 25, wherein administration of the preparation results in a reduced probability of atherosclerosis, myocardial infarct, and/or apoplexy.

28. The method of claim 25, wherein administration of the preparation results in proliferation of T-lymphocytes in the person.

29. The method of claim 25, wherein the person is geriatric.

30. The method of claim 25, wherein the pharmaceutical preparation is administered in food form.
